(19) United States
(12) Patent Application Publication
(10) Pub. No.: US 2014/0030741 A1
(43) Pub. Date: Jan. 30, 2014

(54) In Vitro Method for Determining Presence of Type II Pyrethroids

(71) Applicants: Ki Chang Ahn, Woodland, CA (US); Shirley Jacqueline Gee, Davis, CA (US); Mark Roderick McCoy, Anchorage, AK (US); Zheng Yang, Orlando, FL (US)

(72) Inventors: Ki Chang Ahn, Woodland, CA (US); Shirley Jacqueline Gee, Davis, CA (US); Mark Roderick McCoy, Anchorage, AK (US); Zheng Yang, Orlando, FL (US)

(73) Assignee: GIVAUDAN S.A., Vernier (CH)

(21) Appl. No.: 13/988,546

(22) PCT Filed: Nov. 24, 2011
(86) PCT No.: PCT/EP2011/070906

§ 371 Date: Oct. 3, 2013

Related U.S. Application Data
(60) Provisional application No. 61/416,902, filed on Nov. 24, 2010.

(51) Int. Cl. G01N 33/543

(52) U.S. Cl. CPC G01N 33/54306
USPC 435/7.92; 436/501; 436/173

(57) ABSTRACT

A method of determining the presence of type II pyrethroid compounds in a sample.

This invention relates to a method of determining the presence of type II pyrethroid compounds in a sample.

### BACKGROUND

Type II pyrethroid compounds are a large class of chemical insecticides used extensively for pest control purposes in a wide variety of industries e.g. agriculture, and forestry, as well as in residential settings. They are all synthetic ester compounds and all comprise a cyano (CN) group.

Non limiting examples of type II pyrethroid compounds include acrinathrin, cypermethrin, cyfluthrin, cyhalothrin, deltamethrin, cyphenothrin, fenvalerate, fenpropathrin, flumethrin flucythrinate, fluvalinate, tralomethrin.

Type II pyrethroid compounds are toxic, and in certain cases carcinogenic. This is of particular concern to the food industry because the general population is predominately exposed through the ingestion of contaminated foods and beverages e.g. by consuming contaminated water, fruits & vegetables or extracts thereof that have been treated with such compounds.

Concerns over the contamination of food and water have resulted in the need for food manufacturers, and the suppliers thereof, to test for the presence of type II pyrethroid compounds in water, foodstuffs and food ingredients e.g. additives such as flavourants.

There are a variety of analysis techniques that are currently used to determine the presence of type II pyrethroid compounds in a sample, such as a food or water sample. Common analysis techniques traditionally involve extraction e.g. liquid extraction, followed by gas chromatography (GC), liquid chromatography (LC) coupled with mass spectrometry (MS), or immunochemical analysis.

However, whilst known techniques, such as those exemplified above, are effective when analysis is carried out for only one or two type II pyrethroid compounds, they can become time consuming, complicated, and expensive when determination is required for multiple type II pyrethroid compounds. Further, it is often the case that the specific type II pyrethroid compounds, in some cases to the isomeric level, contaminating a sample must already be known in order to perform an accurate analysis. This is particularly true in the case of immunoassay analysis.

Since many pesticide formulations contain a blend of multiple type II pyrethroid compounds, and since it is not always possible to know to which pesticide formulations, and hence to which type II pyrethroid compounds, a sample, in particular a food and/or water sample, has been exposed, it is often necessary to carry out the analysis for multiple, if not the whole class, of type II pyrethroid compounds, resulting in a labour and time intensive process.

Accordingly it would be beneficial to develop a simple, cost effective, and time efficient method for determining the presence of the entire class of type II pyrethroid compounds in a sample.

### DETAILED DESCRIPTION

The applicant has now found that the presence of type II pyrethroid compounds in a sample can be determined quickly, easily, and economically by a variety of analysis techniques, in particular immunoassays, by subjecting the sample to conditions under which type II pyrethroid compounds would convert to either 3-phenoxybenzoic acid or 4-fluoro-3-phenoxybenzoic acid, and then analysing the sample so treated for the presence of 3-phenoxybenzoic acid and/or 4-fluoro-3-phenoxybenzoic acid.

With the exception of the type II pyrethroid compounds known as flumethrin and cyfluthrin, which may be converted to 4-fluoro-3-phenoxybenzoic acid, all presently known type II pyrethroid compounds may be converted to 3-phenoxybenzoic acid.

The term “sample” as used herein refers to any medium, solid or liquid, that may comprise a type II pyrethroid compound. Non limiting examples of samples include, water, fruit and vegetable extracts e.g. citrus oil, beverages, foodstuffs of all kind, food additives, flavouring agents, fragrances, crop homogenates, house dust, biosolids, and soil samples.

Accordingly in a first aspect of the present invention there is provided an in vitro method for determining the presence of type II pyrethroid compounds in a sample comprising the steps of:
I. Subjecting the sample to conditions under which type II pyrethroid compounds present in the sample will convert to 3-phenoxybenzoic acid or 4-fluoro-3-phenoxybenzoic acid
II. Subsequently analysing the sample for the presence of 3-phenoxybenzoic acid and/or 4-fluoro-3-phenoxybenzoic acid

It may be that a sample subject to such analysis already contains 3-phenoxybenzoic acid and/or 4-fluoro-3-phenoxybenzoic acid. In such a case, the method of determining the presence of type II pyrethroid compounds falls to be determined by measuring said sample before and after conversion to detect any change in concentration of 3-phenoxybenzoic acid and/or 4-fluoro-3-phenoxybenzoic acid.

Any increase in the concentration of the 3-phenoxybenzoic acid and/or 4-fluoro-3-phenoxybenzoic acid, after the conversion of the type II pyrethroid compounds in a sample, would indicate the presence of type II pyrethroids in a sample.

In an aspect of the present invention there is provided an in vitro method for determining the presence of type II pyrethroid compounds in a sample comprising the steps of:
I. Analysing the sample for the presence of 3-phenoxybenzoic acid and/or 4-fluoro-3-phenoxybenzoic acid present in a sample
II. Subjecting the sample to conditions under which type II pyrethroid compounds present in the sample will convert to 3-phenoxybenzoic acid or 4-fluoro-3-phenoxybenzoic acid
III. Subsequently analysing the sample for the presence, or increase in concentration, of 3-phenoxybenzoic acid and/or 4-fluoro-3-phenoxybenzoic acid present in a sample

Type II pyrethroid compounds can be converted to 3-phenoxybenzoic acid or 4-fluoro-3-phenoxybenzoic acid by a variety of known methods using commercially available starting materials, reagents and solvents.

In an illustrative embodiment, the type II pyrethroid compounds are converted to 3-phenoxybenzoic acid or 4-fluoro-3-phenoxybenzoic acid in a process comprising the following steps:
I. Hydrolysis of the type II pyrethroid compounds under basic or acidic conditions to form the corresponding α-cyanoalcohol, α-hydroxy-3-phenoxybenzeneacetonitrile or α-hydroxy-4-fluoro-3-phenoxybenzeneacetonitrile
II. Oxidation of the α-hydroxy-3-phenoxybenzeneacetonitrile or α-hydroxy-4-fluoro-3-phenoxybenzeneacetonitrile, to the corresponding 3-phenoxybenzyl aldehyde(s), and
III. Oxidation to 3-phenoxybenzoic acid, or 4-fluoro-3-phenoxybenzoic acid

Scheme 1 illustrates a process for converting type II pyrethroid compounds to 3-phenoxybenzoic acid, or 4-fluoro-3-phenoxybenzoic acid.

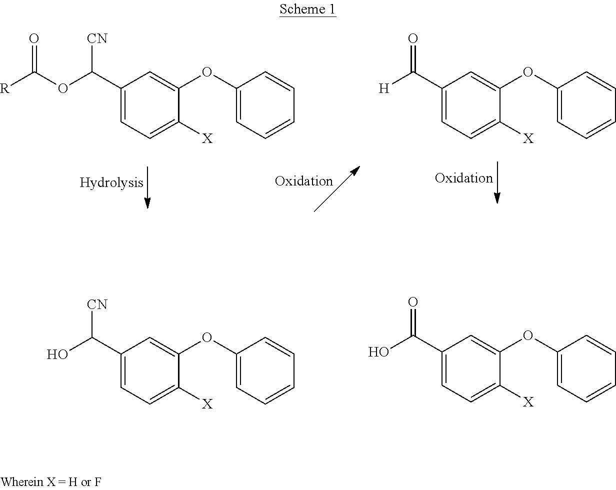

The types and quantities of the hydrolysis and oxidation reagents used should be selected so as to ensure conversion of the type II pyrethroid compounds comprised in a sample.

Factors that may be relevant in deciding on the type and quantities of the hydrolysis and oxidation reagents used may be the relative proportion of the type II pyrethroid compounds typically comprised in a particular type of sample, and the nature of a sample e.g. since citrus oils contain many natural antioxidants it can be difficult to oxidise the type II pyrethroid compounds comprised therein. Further, the insoluble nature of the citrus oil makes hydrolysis difficult.

Typically a large excess of the oxidative and hydrolysing agents in relation to the relative proportions of the type II pyrethroid compounds, typically comprised in a particular type of sample, should be used to ensure conversion of the type II pyrethroid compounds in a sample, in particularly in a citrus oil sample.

Typically samples such as food, water, fruit and vegetable extracts e.g. citrus oil, beverages, foodstuffs of all kind, food additives, flavouring agents, fragrances, crop homogenates, house dust, and soil samples contain these compounds in ppb or ppm concentrations, and thus the person skilled in the art can generally employ the reagents in quantities several orders of magnitude in excess e.g. parts per thousand to ensure the conversion of the type II pyrethroid compounds in a sample.

An additional benefit of using a large excess of the oxidising and hydrolysing agents is that this may ensure the attainment of a pseudo first order reaction rate dependent on the concentration of type II pyrethroid compounds present in a sample.

In an illustrative embodiment the hydrolysis of the type II pyrethroid compounds is performed under basic conditions.

Non-limiting examples of common basic hydrolyzing agents include aqueous metal hydroxides non-limiting examples of which include sodium hydroxide (NaOH), potassium hydroxide (KOH), lithium hydroxide (LiOH), aqueous ammonium hydroxide, triethylamine, N,N-diisopropylethylamine (DIEA), triethanolamine, ammonia gas.

In an illustrative embodiment the hydrolysis of the type II pyrethroid compounds is performed under basic conditions using an aqueous metal hydroxide.

In an illustrative embodiment the hydrolysis of the type II pyrethroid compounds is performed under basic conditions using aqueous NaOH.

Non limiting examples of common oxidizing agents include sodium chlorite, hydrogen peroxide, sodium hypochlorite, silver ion, copper ion, potassium permanganate, ozone, oxygen, halogens non limiting examples of which include chlorine, fluorine, bromine, periodate, chromate reagents non limiting examples of which include chromium trioxide, fenton catalyst and, photooxidation.

In an illustrative embodiment oxidation is carried out using hydrogen peroxide.

Hydrolysis and oxidation, as described herein above, may be carried out independently or simultaneously.

In an embodiment of the present invention hydrolysis and oxidation is carried out simultaneously.

In an illustrative embodiment oxidation and hydrolysis is carried out for a 100 μl sample using 800 μl of a solution of 30% hydrogen peroxide with 0.4M NaOH.

The type II pyrethroid compounds may also be converted to the α-cyanoalcohol α-hydroxy-3-phenoxybenzeneacetonitrile and/or α-hydroxy-4-fluoro-3-phenoxybenzeneacetonitrile, by transesterification.

In an illustrative embodiment, the type II pyrethroid compounds are converted to the α-cyanoalcohol α-hydroxy-3-phenoxybenzeneacetonitrile and/or α-hydroxy-4-fluoro-3-phenoxybenzeneacetonitrile, by transesterification.

As stated herein in relation to the hydrolysing reagents, the reagents selected for transesterification, and quantities in which it is used, will depend on the nature of a sample, and on the relative proportion of the type II pyrethroid compounds typically comprised in a particular type of sample. As for the hydrolysing reagents the transesterification reagent should be added in a large excess e.g. e.g. parts per thousand so as to ensure conversion of the type II pyrethroid compounds in a sample.

In an illustrative embodiment transesterification is carried out using sodium methoxide in the presence of methanol.

Transesterification, and oxidation, as described herein above, may be carried out independently or simultaneously.

Hydrolysis and oxidation may also be carried out enzymatically.

In an illustrative embodiment oxidation and hydrolysis are carried out enzymatically using one or more oxidoreductase, and one or more carboxylesterase enzymes, or an enzymatic system of the foregoing.

The term enzymatic system as used herein refers to a composition comprising more than one enzyme.

Non limiting examples of oxidoreductase enzymes include: NAD(P)+ transhydrogenase (AB-specific), NAD(P)+ transhydrogenase (B-specific), cytochrome b5 reductase leg hemoglobin reductase, NADPH-cytochrome-c2 reductase, NADPH-hemoprotein reductase, 2-hydroxy-1,4-benzoquinone reductase, trimethylamine-N-oxide reductase, aryl-aldehyde dehydrogenase, alcohol dehydrogenase.

Non limiting examples of carboxylesterase enzymes include: methylbutyrase, carboxylic esterase, butyryl esterase, esterase A, esterase B, esterase D, carboxylesterase 1

In an illustrative embodiment hydrolysis is carried out enzymatically using carboxylesterase 1.

In an illustrative embodiment oxidation is carried out enzymatically using aryl-aldehyde dehydrogenase.

In an illustrative embodiment both hydrolysis and oxidation are carried out enzymatically using carboxylesterase 1 and aryl-aldehyde dehydrogenase independently.

In an illustrative embodiment both hydrolysis and oxidation are carried out enzymatically using carboxylesterase 1 and aryl-aldehyde dehydrogenase simultaneously.

The enzymes used in the present invention may be used in immobilised or mobilised form.

In an illustrative embodiment the enzymes are mobilised.

It is generally known that an immobilised enzyme often shows increased resistance to organic solvents as compared to enzymes in the unbound state.

In an illustrative embodiment the enzymes are immobilised.

After oxidation, in particular chemical oxidation, and prior to the subsequent determination of the presence of 3-phenoxybenzoic acid and/or 4-fluoro-3-phenoxybenzoic acid, the reaction mixture may be neutralised, and/or a solid phase extraction(SPE), or liquid-liquid extraction (LLE) step may be carried out,

Neutralisation is particularly useful when the subsequent determination of the presence of 3-phenoxybenzoic acid or 4-fluoro-3-phenoxybenzoic acid, is to be carried out using an immunoassay. This is because it may result in a more sensitive analysis by minimising interference e.g. from the matrix, and/or preventing any denaturing effects of the oxidative agent on the antibody(ies) used in the immunoassay.

SPE or LLE are particularly useful when the subsequent determination of the presence of 3-phenoxybenzoic acid or 4-fluoro-3-phenoxybenzoic acid, is to be carried out using GC or MS because this acts to concentrate and purify a sample for analysis.

It is well within the purview of the person skilled in the art to decide upon whether a neutralisation or SPE step is necessary or desirable depending on how oxidation and hydrolysis have been carried out e.g. enzymatically, and on the analysis technique selected e.g. LC, GC, or immunoassay.

In an illustrative embodiment a neutralisation step is carried out after oxidation and prior to the subsequent determination of the presence of 3-phenoxybenzoic acid or 4-fluoro-3-phenoxybenzoic acid in a sample.

The neutralising agent, and quantities in which it is used, will depend on the specific neutralising agent used, the nature of a sample, and on the quantity and oxidative agent used. It must be selected so as to neutralise the oxidative reagent

Non limiting examples of common neutralising agents include ascorbic acid, a platinum catalyst, zinc metal, sodium metabisulfite, hydrogen gas, silver metal, palladium metal, manganese oxide, catalase.

It is well within the purview of the persons skilled in the art to decide upon specific reagents and quantities of the neutralising agent on the basis of the oxidative reagent and the quantity in which it has been used. The pH of the reaction mixture may be used to indicate when a sufficient amount of a neutralising agent has been added.

In an illustrative embodiment neutralisation is carried out using a platinum catalyst.

In an illustrative embodiment neutralisation is carried out, following the oxidation of the type II pyrethroid compounds in a 100 μl sample using 800 μl of a solution of 30% hydrogen peroxide, using a 0.5% platinum on alumina.

In an illustrative embodiment a SPE step is carried out after oxidation and prior to the subsequent determination of the presence of 3-phenoxybenzoic acid and/or 4-fluoro-3-phenoxybenzoic acid in a sample.

In a particular embodiment of the present invention the sample is a citrus oil sample.

In an illustrative embodiment of the present invention the sample is citrus oil and hydrolysis is carried out using, sodium hydroxide, oxidation using hydrogen peroxide, and neutralisation using a platinum catalyst on a solid support.

In another illustrative embodiment of the present invention the sample is water spiked with citrus oil and hydrolysis is carried out using, sodium hydroxide, oxidation using hydrogen peroxide, and neutralisation using a platinum catalyst on a solid support.

In an illustrative embodiment a 100 μl citrus oil sample was hydrolysed and oxidised using 800 μl of a solution of 30% hydrogen peroxide, and neutralised using a 0.5% platinum on alumina pellet.

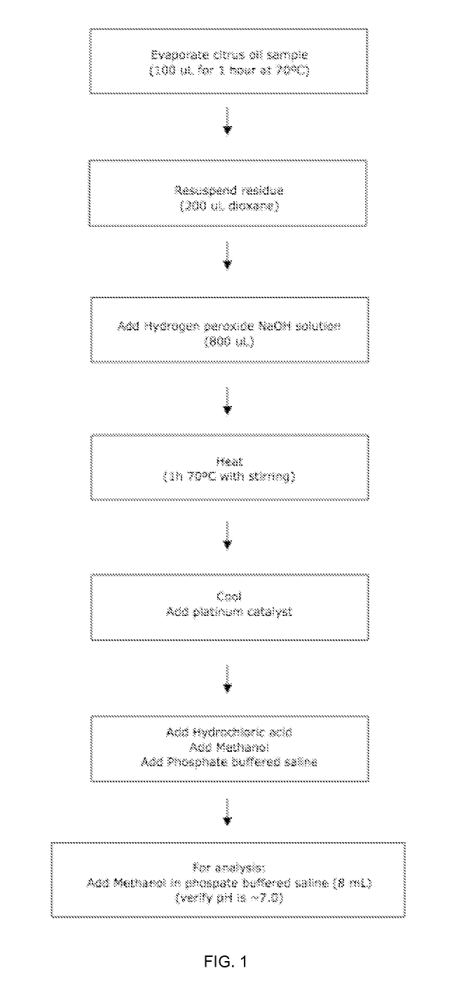

A process for the conversion of type II pyrethroid compounds, to 3-phenoxybenzoic acid or 4-fluoro-3-phenoxybenzoic acid, comprised in a citrus oil sample is illustrated in FIG. 1.

The presence of 3-phenoxybenzoic acid and/or 4-fluoro-3-phenoxybenzoic acid prior or after the conversion of the type II pyrethroid compounds in a sample, can be determined by any means known to those skilled in the art, for example immunoassay, GC, LC, MS, high pressure liquid chromatography (HPLC), and combinations thereof.

In an illustrative embodiment of the present invention the presence of 3-phenoxybenzoic acid and/or 4-fluoro-3-phenoxybenzoic acid prior or after the conversion of the type II pyrethroid compounds is determined by an immunoassay.

Prior to analysis via immunoassay the pH of the sample may be adjusted to a pH within the range of 6 to 9.

Any type of immunoassay capable of determining the presence and/or concentration of 3-phenoxybenzoic acid and/or 4-fluoro-3-phenoxybenzoic acid may be used. Non limiting examples of possible immunoassays include, competitive immunoassay, non-competitive immunoassay, radioimmunoassay, fluorescent immunoassay, magnetic immunoassay, PCR immunoassay, electrochemiluminescent immunoassay, and enzyme linked immunosorbent assay (hereinafter ELISA).

In an illustrative embodiment of the present invention the presence of 3-phenoxybenzoic acid and/or 4-fluoro-3-phenoxybenzoic acid prior or after the conversion from the type II pyrethroid compounds is determined by an ELISA.

Any enzyme, antigen, and antibody combination capable of determining the presence of 3-phenoxybenzoic acid and/or 4-fluoro-3-phenoxybenzoic acid may be used in the ELISA.

Non limiting examples of possible enzymes include: Peroxidase, Phosphatase, and B-galactosidase.

In an illustrative embodiment peroxidase is used in the ELISA to determine the presence of 3-phenoxybenzoic acid and/or 4-fluoro-3-phenoxybenzoic acid in a sample.

Non limiting examples of possible antigens include: a 3-((2-oxoethoxy)ethoxy)phenoxybenzoic acid-thyroglobulin conjugate as an immunogen, and a 3-PBA-bovine serum albumin as a coating antigen in this competitive format.

Shan, G.; Huang, H.; Stoutamire, D. W.; Gee, S. J.; Leng, G.; Hammock, B. D. Chem. Res. Toxicol. 2004, 17, 218-225, incorporated herein by reference, contains more information on these antigens.

Any antibody reactive with 3-PBA may be used in the ELISA to determine the presence of 3-phenoxybenzoic acid and/or 4-fluoro-3-phenoxybenzoic acid in a sample.

In an illustrative embodiment the antibody Rabbit anti-3-PBA #294 is used in the ELISA. Shan, G.; Huang, H.; Stoutamire, D. W.; Gee, S. J.; Leng, G.; Hammock, B. D. Chem. Res. Toxicol. 2004, 17, 218-225, incorporated herein by reference, contains more information on this antibody.

The methods herein disclosed maybe used to detect type II pyrethroid compounds present in a sample in a concentration of 1 ppm or more, 0.5 ppm or more, 1 ppb or more, 0.5 ppb or more. The lower detection limit depending on the nature of a sample in question i.e. lower detection limits are higher for citrus oil samples in comparison to water samples because of the complex composition of citrus oil and the difficulties in hydrolysing and oxidising the type II pyrethroid compounds comprised therein.

In an illustrative embodiment the sample is water and the lower detection limit is 0.5 ppb

In an illustrative embodiment the sample is citrus oil and the lower detection limit is 0.5 ppm

The invention will now be described in further detail by way of the following non limiting examples.

### Example 1

### Conversion of Type II Pyrethroid Compounds in Orange Oil Using Hydrogen Peroxide as Oxidant and a Platinum Catalyst as the Neutralizer

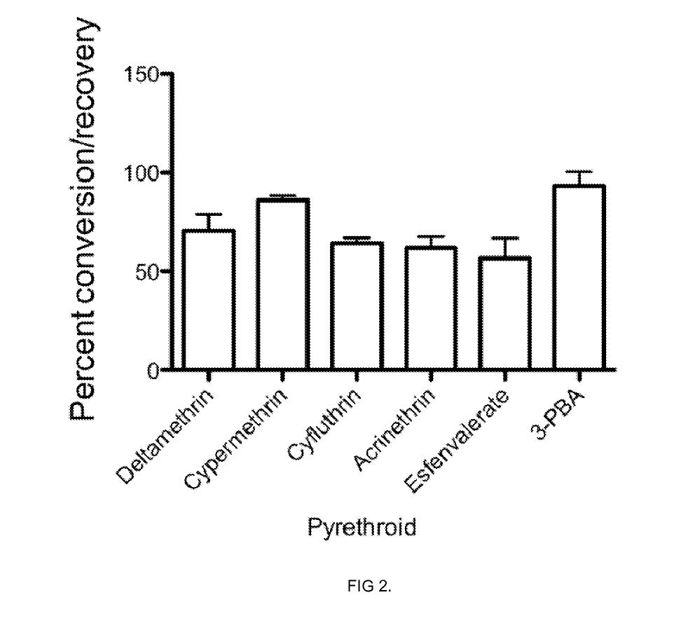

Orange oil was spiked with a final concentration of 10 μM of the indicated pesticide and all samples were run in triplicate. 100 μL of the orange oil sample was pipetted into a glass vial before it was volatilized for 1 hour under a steady stream of compressed air. Samples then had 200 μL of dioxane and 800 μL of 30% w/v hydrogen peroxide with 0.4 N NaOH added. Samples were then incubated with stirring for 1 hour at 50° C. Samples were then neutralized by addition of a 0.5% platinum on alumina pellet, which was allowed to incubate without stirring at 25° C. for 2 hours to complete the neutralization. Samples were then diluted 200× into 10% MeOH 90% 0.1 M phosphate buffer prior to analysis by ELISA. Results can be seen in FIG. 2

### Example 2

### Conversion of Type II Pyrethroid Compounds from Orange Oil Using Sodium Chlorite as Oxidant and Ascorbic Acid as the Neutralizer

Orange oil was spiked with a final concentration of as indicated with the pesticides and all samples were run in triplicate. 100 μL of the orange oil sample was pipetted into a glass vial before it was volatilized for 1 hour under a steady stream of compressed air. Samples then had 200 μL of dioxane and 800 μL of 10% w/v sodium chlorite with 0.4 N NaOH added. Samples were then incubated with stirring for 1 hour at 75° C. Samples were then cooled and analyzed in two separate ways. The samples that were analyzed by ELISA had 1.0 mL of 1.0 M ascorbic acid added. The pH was modified up to ˜7 by addition of NaOH before the samples were diluted 400× with 10% MeOH 90% 0.1 M phosphate buffer prior to analysis by ELISA. The samples that were analyzed by GC and LC/MS had their pH adjusted with HCl down to 5 and diluted with 1.0 mL of 0.2 M sodium acetate buffer pH 4.5. These samples were then cleaned up using a Strata-screen A mixed mode SPE column before being analyzed by LC/MS and GC/MS. Ahn K C, Lohstroh P, Gee S J, Gee N A, Lasley B, Hammock B D. Anal Chem. 2007 79(23):8883-8890 incorporated herein by reference, contains more information on this SPE method.

Results can be seen in table 1.

TABLE 1

| | | | | | | Spike | |
|---|---|---|---|---|---|---|---|
| ELISA | SD | GC/MS | SD | LC/MS | SD | level | Pyrethroid |
| 5.8 | 0.5 | 12.9 | 2.1 | 23.2 | 3.4 | 10.6 | Deltamethrin |
| 5.8 | 0.9 | N/A | | N/A | | 11.3 | beta-cyfluthrin |
| 5.6 | 0.7 | 6.9 | 1.0 | 15.5 | 2.8 | 9.5 | (4.6) deltamethrin, |
| | | | | | | | (4.9) acrinethrin |
| 6.6 | 0.9 | 6.7 | 1.2 | 19.5 | 3.8 | 11.3 | (3.7) deltamethrin, |
| | | | | | | | (3.8) acrinethrin, |
| | | | | | | | and (3.8) lambda- |
| | | | | | | | cyhalothrin |
| 0.9 | 0.1 | <LOQ | | 5.5 | 0.4 | 2.1 | Deltamethrin |
| 1.0 | 0.2 | N/A | | N/A | | 2.3 | beta-cyfluthrin |
| 1.5 | 0.3 | 3.4 | 0.1 | 6.7 | 0.5 | 1.9 | (0.92) deltamethrin, |
| | | | | | | | (0.98) acrinethrin |
| 1.5 | 0.2 | 4.3 | 0.4 | 8.3 | 0.5 | 2.3 | (0.74) deltamethrin, |
| | | | | | | | (0.76) acrinethrin, |
| | | | | | | | and (0.76) lambda- |
| | | | | | | | cyhalothrin |

### Example 3

### Conversion of Pyrethroid Compounds from Orange Oil Using Sodium Chlorite as Oxidant and Ascorbic Acid as the Neutralizer

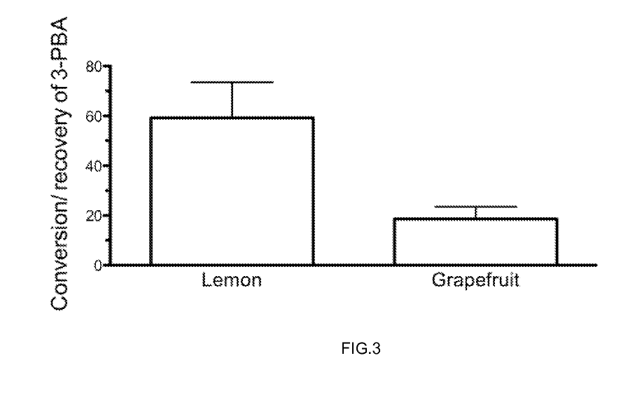

Grapefruit and Lemon oil was spiked with a final concentration of 2.5 ppm deltamethrin and all samples were run in triplicate. 100 μL of the citrus oil sample was pipetted into a glass vial before it was volatilized for 1 hour under a steady stream of compressed air. Samples then had 200 μL of dioxane and 800 μL of 10% w/v sodium chlorite with 0.4 N NaOH added. Samples were then incubated with stirring for 1 hour at 75° C. Samples were then cooled and analyzed in two separate ways. The samples had 1.0 mL of 1.0 M ascorbic acid added. The pH was modified up to ˜7 by addition of NaOH before the samples were diluted 400× with 10% MeOH 90% 0.1 M phosphate buffer prior to analysis by ELISA. Results can be seen in FIG. 3

1. An in vitro method for determining the presence of type II pyrethroid compounds in a sample comprising the steps of:
I. Subjecting the sample to conditions under which type II pyrethroid compounds present in the sample will convert to 3-phenoxybenzoic acid or 4-fluoro-3-phenoxybenzoic acid,
II. Subsequently analysing the sample for the presence of 3-phenoxybenzoic acid and/or 4-fluoro-3-phenoxybenzoic.

2. The method according to claim 1 additionally comprising the step of first determining the presence of 3-phenoxybenzoic acid and/or 4-fluoro-3-phenoxybenzoic acid in the sample before subjecting the sample to conditions under which type II pyrethroid compounds will convert to 3-phenoxybenzoic acid or 4-fluoro-3-phenoxybenzoic acid.

3. The method according to claim 1 wherein the conditions under which type II pyrethroid compounds will convert to 3-phenoxybenzoic acid, or 4-fluoro-3-phenoxybenzoic acid comprise the following steps in sequence:
I. Hydrolysis of the type II pyrethroid ester group under basic or acidic conditions to form an α-cyanoalcohol α-hydroxy-3-phenoxybenzeneacetonitrile, or α-hydroxy-4-fluoro-3-phenoxybenzene-acetonitrile,
II. Oxidation of α-hydroxy-3-phenoxybenzeneacetonitrile, or α-hydroxy-4-fluoro-3-phenoxybenzeneacetonitrile to 3-phenoxybenzoic acid or 4-fluoro-3-phenoxybenzoic acid,
III. Optional neutralisation of an oxidative agent used in step II.

4. The method according to claim 3 wherein the hydrolysis is performed under basic conditions.

5. The method according to claim 4 wherein the hydrolysis is performed using aqueous sodium hydroxide.

6. The method according to claim 3 wherein the oxidation is performed using hydrogen peroxide.

7. The method according to claim 3 wherein the hydrolysis is carried out enzymatically.

8. The method according to claim 3 wherein the oxidation is carried out enzymatically.

9. The method according to claim 3 wherein the hydrolysis and the oxidation are carried out simultaneously.

10. The method according to claim 3 wherein the neutralisation is performed using a platinum catalyst.

11. The method according to claim 1 wherein determining the presence of the 3-phenoxybenzoic acid, and/or 4-fluoro-3-phenoxybenzoic acid, is carried out by GC, LC, HPLC MS, an immunoassay, or a combination of the foregoing.

12. The method according to claim 1 wherein determining the concentration of the 3-phenoxybenzoic acid, or 4-fluoro-3-phenoxybenzoic acid, is carried out by an ELISA.

13. The method according to claim 1 wherein the sample is selected from the group consisting of: water, fruit extracts, vegetable extracts, beverages, foodstuffs, food additives, flavouring agents, fragrances, crop analysis, house dust, biosolids, and soil samples.

14. The method according to claim 1 wherein the sample is a citrus oil sample.
